(19) United States
(12) Patent Application Publication
(10) Pub. No.: US 2006/0270976 A1
(43) Pub. Date: Nov. 30, 2006

(54) Steerable catheter

(75) Inventors: James D. Savage, Port Jefferson Station, NY (US); Jaime Merino, Elmont, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: ProRhythm, Inc., Ronkonkoma, NY (US)

(21) Appl. No.: 11/439,568

(22) Filed: May 23, 2006

Related U.S. Application Data
(63) Continuation of application No. 11/141,426, filed on May 31, 2005.

(51) Int. Cl. A61M 31/00

(52) U.S. Cl. 604/95.04

(57) ABSTRACT

A steerable catheter has a steering portion incorporating a spear cut junction of catheter material, thereby providing for a gradual change in flexibility in the steering portion. A guide tube and spring may be located inside a pull wire lumen of the catheter, with the spring distal to the guide tube so that the spring is disposed at least partially within steering section. A steering mechanism moves the pull wire linearly in order to steer the catheter.

### CROSS REFERENCE TO RELATED APPLICATIONS

This application is a continuation of application Ser. No. 11/141,426, filed May 31, 2005, the disclosure of which is hereby incorporated herein by reference.

### FIELD OF THE INVENTION

The present invention generally relates to steerable catheters, and more particularly to the steering, responsiveness and kink resistance aspects of steerable catheters.

### BACKGROUND OF THE INVENTION

Steerable, or deflectable, catheters are widely used in medical procedures to gain access to, and operate on, interior regions of the body. Such catheters have a distal end which can be remotely manipulated via a proximally located steering mechanism. In a typical medical procedure, the steering mechanism is located outside of the patient's body, and is manipulated in order to steer the distal end of the catheter to a desired location within the body. A steering catheter is disclosed, for example, in U.S. patent application Ser. No. 10/783,310, published as U.S. Published Patent Application No. 2004/0176757, entitled “Cardiac Ablation Devices,” the full disclosure of which is hereby incorporated by reference herein.

The catheter's distal end may carry instrumentation to facilitate viewing and/or performing various surgical procedures, such as surgical ablation, at the remote location in the patient. A surgical ablation catheter, such as one using ultrasound to ablate tissue, is disclosed in the aforesaid U.S. Published Patent Application 2004/0176757 and in U.S. Pat. No. 6,635,054, the full disclosures of which are hereby incorporated by reference herein.

It is important that a physician can precisely and reliably control the movement of the catheter, especially during procedures that require positioning the catheter within the heart. In cardiac procedures, for example, a physician navigates the catheter through the patient's vasculature into the interior region of the heart that is to be examined and/or treated. Once the distal end of the catheter has reached a desired location, the catheter is further manipulated at that location in accordance with the particular procedure that is to be carried out. For example, in certain preferred embodiments as set forth in the aforementioned '054 patent and '757 publication, the ablation device includes an ultrasonic transducer and a reflector structure adapted to direct ultrasonic waves emitted by the transducer forwardly and outwardly from the axis of the device into a ring-like ablation region surrounding the axis and distal to the device. In certain procedures using such ablation devices, the catheter tip may be bent to a desired angle, and the catheter rotated so as to position the ablation device, and hence the ring-like ablation region, such that the ring-like ablation region extends around the ostium of a pulmonary vein. For example, in treatment of atrial fibrillation using such devices, an especially sharp bend may be required to position the ablation device in alignment with the ostium of the right inferior pulmonary vein.

A steering catheter typically has at least one tendon wire, or pull wire, located in a lumen somewhere in its periphery. This longitudinally running wire is commonly anchored at the distal end of the catheter, and connected to the steering mechanism at the proximal end of the catheter. The steering mechanism typically has an interface section, such as a slide-handle or wheel, that allows the physician to exert an axial pulling force on the wire. As the wire is pulled proximally, the anchored distal end of the wire deflects, thus causing the distal end of the catheter to bend. The bending of the catheter away from center occurs towards the direction of the peripheral location of the tendon wire.

A catheter that only has one tendon wire is only able to bend in one direction, i.e., to one side of the proximal-to-distal axis of the catheter. This is known as uni-directional steering. However, since a catheter can be rotated, any point surrounding the distal end of the catheter may be reached by bending the catheter tip and rotating the catheter. Multi-directional steering involves having two or more peripherally located tendon wires that facilitate bending the catheter in two or more directions.

One common drawback of steering mechanisms is that the connection point of the wire to the steering mechanism is not linearly aligned with the entry point of the wire into the body of the catheter. Such misalignment can cause the pull wire to bend. Another common drawback is that the wire is run over a sheave or pulley for alignment and manipulation purposes. Such sheaves and pulleys add complexity and friction. Moreover, misalignment, or the use of sheaves and pulleys, can cause the pull wire to fatigue and can ultimately lead to premature failure of the pull wire. Thus, it is desirable to have a steering mechanism that maintains the pull wire aligned with its entry point into the body of the catheter, and does not require pulling the wire over a guide surface, such as a pulley or sheave.

Occasionally, upon exerting a pulling force on a pull wire, the catheter body may bow into a “C” shape before the distal end begins to deflect and steer. Undesirably, this occurrence requires the physician to pull on the pull wire even further requiring increasingly high forces in order to get the distal end of the catheter to deflect, or steer. Additionally, the bowing effect imparts unwanted and unexpected movement to the catheter in areas other than the distal end. These undesirable effects increase in significance as the diameter of the catheter increases, and can reach the limits of user acceptance. Thus it is desirable to have a catheter design that provides an efficient deflection mechanism and catheter structure that enables minimal force to deflect the catheter, predictable movement of the catheter, and minimizes or alleviates unwanted deflection in the body of the catheter.

Typically, the distal steering end of a catheter is comprised of a softer, more flexible material, while the body of the catheter is comprised of a more rigid material. Commonly, the transition area of the catheter where these two materials meet is prone to kinking, or collapse, when the distal end of the catheter is steered. Kinking can interfere with accurate steering of the distal end of the catheter, and can close lumens within the catheter, and can otherwise render the catheter non-functional. Additionally, because the kink creates an area of localized drastic material deformation, the pull wire may cut through the kinked material and exit the catheter at that location. As such, it is desirable to have a catheter with a transition section that resists kinking and exiting of the pull wire.

### SUMMARY OF THE INVENTION

One aspect of the present invention provides a catheter. The catheter according to this aspect of the invention desirably includes an elongated catheter body having a proximal-to-distal axis. The body includes a proximal shaft portion and a distal shaft portion that is more flexible than the proximal shaft portion, said proximal and distal shaft portions joining on another at a junction. Most preferably, the junction is generally oblique to the proximal-to-distal axis so that said proximal shaft portion terminates at an apex on a first side of said catheter and at a base on a second side diametrically opposite to said first side, said base being proximal to said apex. This arrangement can provide a gradual transition in flexibility to minimize kinking and stress concentration when the catheter is bent, as well as a reduced deflection radius to better access tightly confined target areas. Most preferably, the catheter includes a steering mechanism arranged to bend the catheter toward the first or apex side, so that the apex of the transition lies on the concave side of the bend formed when the steering mechanism is actuated.

A further aspect of the present invention provides a catheter incorporating an elongated catheter body defining a pull wire lumen. The catheter further includes a guide tube and a coil spring located inside the pull wire lumen, with the spring being distal to said guide tube. The pull wire runs through the guide tube and coil spring. The guide tube and spring provide a low-friction environment for the pull wire, and minimize binding. The guide tube and spring also resist any tendency of the pull wire to cut through the catheter body.

Yet another aspect of the invention provides a steerable catheter unit. The unit desirably includes a housing which may be in the form of a handle and a catheter body having a main portion projecting in a distal direction from the housing. The catheter body may be provided with a flexible guide tube extending in said main portion of said catheter body. The guide tube desirably has a proximal section projecting out of the catheter body within the housing. Here again, a pull wire is slidably disposed within said guide tube, the pull wire having a distal end extending distally from said guide tube and connected to said catheter. The pull wire has a proximal end extending proximally from the guide tube.

An outer movement element is movably mounted on an outside of the housing. An inner movement element is disposed within the housing and connected to said proximal end of the pull wire. The inner movement element most preferably is in telescopic relation to said proximal end of the guide tube. The inner movement element is linked to the outer movement element so that the inner movement element, and hence the pull wire, can be moved by moving the outer movement element. The telescopic arrangement of the inner movement element and guide tube provides a straight path for the proximal end of the pull wire, which minimizes friction between the pull wire and the proximal end of the guide tube. The proximal section of the guide tube desirably is substantially straight as well. This arrangement can provide a substantially straight path for the pull wire within the housing.

### BRIEF DESCRIPTION OF THE DRAWINGS

FIG. 1 is a diagrammatic view depicting a catheter according to one embodiment of the invention, where a portion of the catheter is in a section of the heart.

FIG. 2 is a diagrammatic view of a segment of the catheter depicted in FIG. 1.

FIG. 3 is a fragmentary, sectional view of the catheter segment depicted in FIG. 2.

FIG. 4 is a diagrammatic view of the catheter segment depicted in FIG. 2.

FIG. 5 is a fragmentary diagrammatic sectional view of a catheter according to a further embodiment of the invention.

FIG. 6 is a diagrammatic view of a segment of the catheter according to yet another embodiment of the invention.

FIG. 7 is a fragmentary, sectional view of the catheter segment depicted in FIG. 6.

FIG. 8 is a close-up, sectional view of area 8 of the catheter segment depicted in FIGS. 6 and 7.

FIG. 9 is a diagrammatic cutaway view of a steering handle according to yet another embodiment of the invention.

FIG. 10 is a further diagrammatic view of the steering handle depicted in FIG. 9.

FIG. 11 is a fragmentary sectional view along line 11-11 in FIG. 10.

FIG. 12 is a diagrammatic view of the catheter depicted in FIG. 1, and an ablation device, in accordance with yet another embodiment of the invention.

FIG. 13 is a schematic view depicting certain geometric relationships between the catheter depicted in FIG. 10 and a portion of the heart wall.

### DETAILED DESCRIPTION

In order to define spatial relationships between the parts of a medical instrument, as used in this disclosure, the term “distal” refers to an area which is closer to the body of the patient, or inserted first into the body of the patient and penetrates to the greatest depth within the body. The term “proximal” refers to an area opposite the distal area.

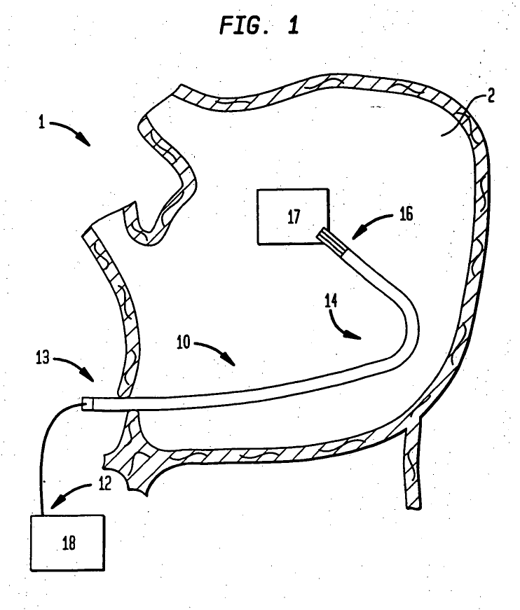

Referring to FIG. 1, the apparatus according to one embodiment of the invention is a catheter, generally identified as 10, having a generally depicted steering device 18 on its proximal end 12, an insertable catheter portion 13 which gets inserted into a subject, such as into a chamber 2 of the subject's heart 1, a steering portion 14 and a distal end 16.

The distal end 16 of the catheter 10 may house various tools and instruments 17 that facilitate the execution of different procedures, such as surgical ablation for example, at a target location in a subject. The distal end 16 is manipulated via the steering device 18, which will be described in more detail, below. Generally, steering device 18 allows a user to pull on at least one pull wire in the catheter 10, which causes the steering portion 14 of the catheter 10 to bend, thus resulting in movement of the distal end 16, as depicted in FIG. 1, for example. Additionally, rotation of the steering device 18, allows the distal end 16 to rotate. Thus, if the distal end 16 is bent and rotated, it will sweep in a circular motion, thereby allowing a user to direct it to any desired point within a subject.

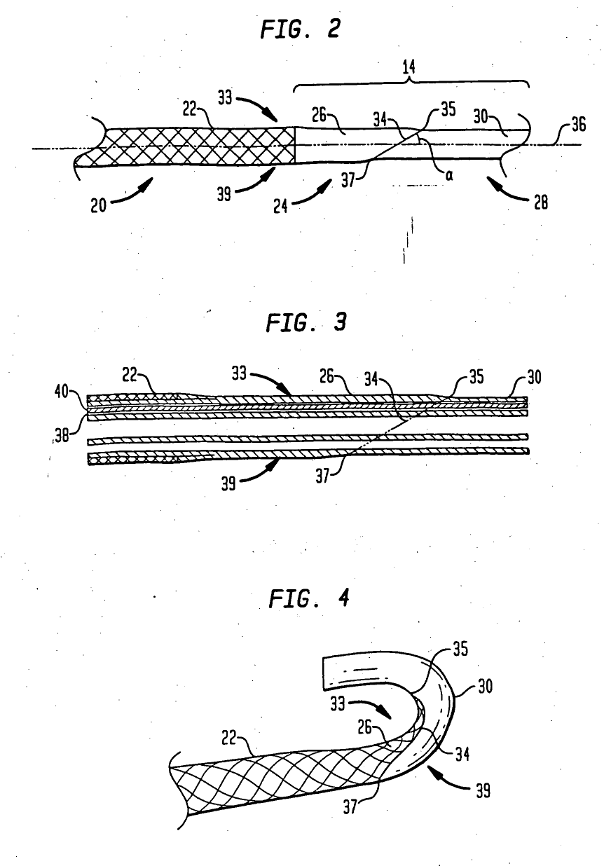

Referring to FIGS. 2-4, catheter 10 includes a main shaft 22, a transition shaft 26 extending distally from the main shaft, and a distal shaft 30 extending distally from the transition shaft. Main shaft 22 is relatively stiff, whereas transition shaft 26 is more flexible in bending than the main shaft, and distal shaft 30 is even more flexible than the transition shaft. Thus, the proximal portion 20 of the catheter, made up of the main shaft 22 and transition shaft 26, is stiffer than the distal shaft 30. In the embodiment depicted, the main shaft 22 has the largest outside diameter and transition shaft 26 has a slightly smaller outside diameter, whereas distal shaft 30 has a still smaller outside diameter. Stated another way, one or more of the cross-sectional dimensions of the transition shaft, in directions transverse to the axis 36 of the catheter, decreases from main shaft 22 to transition shaft 26, and further decreases from the transition shaft 26 to distal shaft 30. Also, the main shaft 22 may be formed from a relatively high-durometer material and may include a reinforcing material such as a braided reinforcement incorporated within the tubing. Transition shaft 24 may be formed from the same material as the main shaft but without the reinforcement, or may be formed from a softer material than the main shaft. Distal shaft 30 may be formed from a softer, lower-durometer material than the proximal shaft. The shafts typically are formed from polymeric materials such as Pebax™, manufactured by Autofina. Although the main shaft, transition section and distal shaft are referred to separately herein, it should be appreciated that these shafts together form a unitary catheter body. The transition shaft 24 and distal shaft 28 cooperatively constitute the steering portion 14 of the catheter.

The catheter 10 defines a pull wire lumen 38 extending through the catheter body in a position offset from the central axis 36 of the catheter, so that the pull wire lumen 38 lies near the periphery of the catheter closer to a first side 33 (the side of the catheter toward the top of the drawing in FIG. 3) than to the opposite, second side 39. The catheter also defines additional lumens which may be used to convey fluids or instruments through the catheter, or which may house additional structures (not shown) such as electrical wires or optical fibers connected to the instrument 17 on the distal end of the catheter.

A pull wire 40 is located in pull wire lumen 38. The material for the pull wire 40 may be any suitable material usable with a catheter, such as stainless steel wire. The pull wire 40 is connected at its proximal end to the steering device 18, and anchored at its distal end to the distal shaft 30 of the catheter 10 or to the instrument 17 mounted on the distal end of the catheter. Thus, the pull wire 40 passes through the steering portion 14 of the catheter 10 and is mechanically connect to the catheter within or distal to the steering portion. Thus, when tension is applied to pull wire 40, the catheter body will tend to bend toward first side 33, into a curved configuration as seen in FIG. 1. Because the steering portion 14 of the catheter, and particularly distal shaft 30 is more flexible than the other regions of the catheter, such bending occurs principally in the steering portion 14.

To maximize deflection and minimize the deflection radius without kinking at the juncture of the proximal portion 20 of the catheter (main shaft 22 and transition section 26) and the distal portion of the catheter constituted by shaft 30, the junction between the proximal and distal portions is formed so that this junction is oblique to the proximal-to-distal axis 36 of the catheter. The transition 34 has an angle α, identified in FIG. 2, that is less than 90° but greater than 0° with respect to the proximal-to-distal catheter axis 36. Thus, the transition section 24 of the proximal shaft portion 20 terminates in a “spear cut” configuration, so that the transition section terminates at an apex 35 on the first side 33 of the catheter and at a base 37 on the second side 39 of the catheter, diametrically opposite from the base. Stated another way, the stiffer proximal portion 20 of the catheter extends further in the distal direction on the first side than on the second side.

As discussed above, tension applied to the pull wire 40 tends to bend the steering section of the catheter toward the first side 33. Thus, the apex 35 of the transition, and hence the distally projecting side of transition section 26, will lie on the concave side 33 of the steering section 14 when the pull wire 40 is pulled and the steering section 14 is bent. The oblique transition provides a gradual transition stiffness near the proximal end of the steering section 14, and thus near the proximal end of the bend, thereby reducing the potential for kinking. Tension in the pull wire 40 tends to cause the pull wire to cut through the material of the catheter on the first side 33, i.e., on the concave side of the bend. The distally-projecting apex of the transition on the first side of the catheter provides additional reinforcement which helps prevent the pull wire 40 from cutting through the catheter on this side.

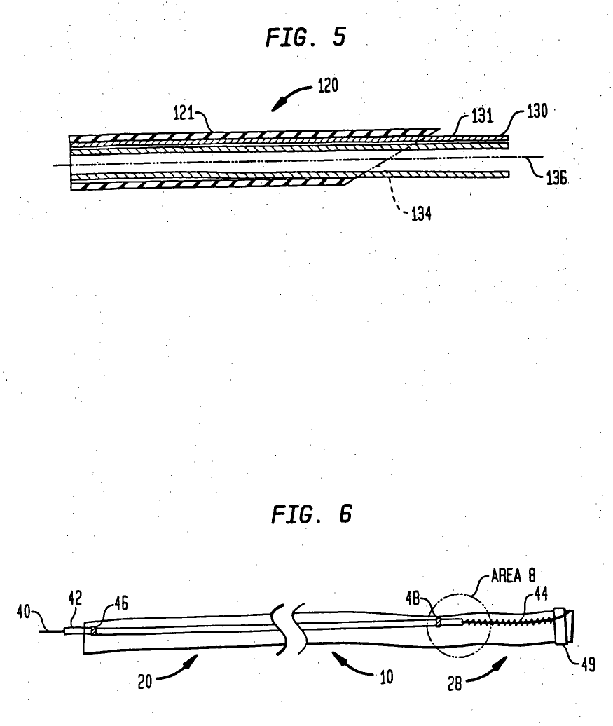

In a variant of the structure discussed above with reference to FIGS. 1-4, a unitary tubular member 131 (FIG. 5) forms the distal section 130 of the catheter and also extends into the proximal portion 120 of the catheter. The catheter includes an outer reinforcing member 121 extending around the tubular member in proximal portion 120. Thus, the proximal section 120 includes both the reinforcing member 121 and that portion of unitary member 131 disposed within the reinforcing member. The transition 134 between the proximal portion 120 and distal portion 130 is defined by the distal end of the reinforcing member. Here again, the transition is oblique to the proximal-to-distal axis 136 of the catheter, so that the reinforcing member 121, and hence the proximal section 120, has an apex 135 on the first side of the catheter and a base 137 on the second, opposite side.

The structure of FIG. 5 does not include a transition section. Likewise, the transition section 24 of the catheter 10 discussed above with reference to FIGS. 1-4 may be omitted, so that the transition between the proximal and distal portions is provided directly between the main shaft 22 and distal shaft 30. Alternatively, more than one transition section can be used. In a further variant, the transitions between sections, such as between main shaft 22 and transition section 26 (FIGS. 2-4) may include oblique transitions.

Thus, an oblique or “spear cut” transition similar to transition 34 or 124 may be located on any catheter section that transitions into another section. Additionally, the oblique transition need not have a straight line profile, as depicted in FIGS. 2 and 3, between its apex 35 and base 37, but may have any profile that extends between the apex 35 and base 37.

A catheter according to a further embodiment of the invention, shown in FIGS. 6-8, has a catheter body similar to that shown and discussed above with reference to FIGS. 2-4. In this embodiment, preferably, the pull wire 40 is situated inside a guide tube 42 which in turn is disposed in the pull wire lumen 38. The guide tube 42 can be made of a material such as, for example, stainless steel or other metal, or from a hard polymeric material, such as polyimide or PTFE, or from a polymer lined metal tube, such as a Teflon lined stainless steel tube, the latter being preferred. The guide tube may be a tube of the type commonly used to fabricate hypodermic needles, i.e., a stainless steel tube having an outside diameter of about 0.050 inches or less, and more preferably about 0.018 inches or less. Such tubing is sometimes referred to as “hypotube.” Merely by way of example, the guide tube may be a 26 gauge stainless steel hypodermic tube, with a nominal outside diameter of 0.0183 inches and a nominal wall thickness of 0.004 inches. The guide tube desirably provides and exhibits high strength and resiliency that resists compression.

Preferably, the guide tube 42 extends through the majority of the length of the proximal portion 20 of the catheter from the steering device 18 (FIG. 1) to the steering portion 14. Within the catheter's proximal portion 20, the guide tube 42 may be anchored in the pull wire lumen 38 at two anchor points 46 and 48. Anchoring may be achieved by using an adhesive, by melting a localized area of catheter material in the lumen onto the guide tube 42, or by any other suitable method. Preferably, the proximal anchor point 46 is formed with an adhesive, and the distal anchor point 48 is formed by melting. The anchor points 46, 48 prevent the guide tube 42 from freely traveling within the lumen 38 during the catheter's operation. This helps establish fixed locations of specific performance properties of the catheter 10, thus enabling more predictive behavior during the catheter's operation.

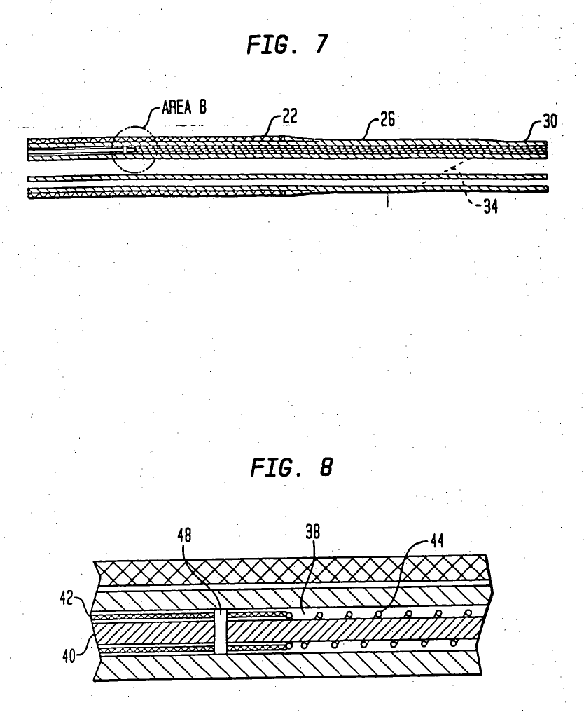

Preferably, the distal anchor point 48 for the guide tube 42 is in an area 8 that is in or just proximal to the steering portion 14 of the catheter 10, and proximal to the transition 34. The location of the distal anchor point 48, as well as the location of the distal end of the hypotube, is such that they tend not to affect the bendability characteristics of the steering portion 14. FIG. 7 is a close-up view of area 8.

The guide tube 42 provides an increased level of rigidity to the proximal portion 20 of the catheter, as well as a low-friction surface surrounding the pull wire 40. The increased rigidity lowers the tendency for the proximal portion 20 of the catheter body to deflect, or compress, when the pull wire 40 is pulled. Stated another way, the guide tube further increases the difference in stiffness between the main shaft 22 and the distal shaft 30. Additionally, the hard, low-friction surface of the guide tube minimizes the tendency for the pull wire 40 to drag a surrounding surface that it may contact while it is being pulled. Minimizing drag also helps to reduce the pull forces needed to deflect the tip, as well as the tendency for the proximal portion 20 of the catheter to bow when the pull wire 40 is pulled.

The length of the guide tube 42 may be shorter than that described above. Alternatively, the guide tube 42 may extend through the steering portion 14 to the pull wire ring 49 that anchors the pull wire 40 within the distal portion 28 of the catheter 10, so long as it can repeatedly bend without kinking when the steering portion 14 is bent, and return to its straight shape when the steering portion 14 is straightened. Additionally, more than two anchor points may be formed between the guide tube 42 and the catheter 10.

As also shown in FIGS. 6-8, a coil spring 44 is located distal to the guide tube 42 in pull wire lumen 38, so that the coil spring surrounds the pull wire 40. Preferably, the spring extends through at least the major portion of the steering portion 14, and most desirably extends from the guide tube 42 to the point where the pull wire is attached to the distal shaft or instrument. In this embodiment, the distal shaft has an anchor ring 49, and the pull wire is attached to the ring.

The spring 44 may be of any suitable material, but most preferably is formed from a metallic material such as stainless steel. The spring desirably has characteristics similar to those of the hypotube 42, such as a low friction surface. Advantageously, a coil spring does not tend to kink when bent. Thus, the placement of the spring 44 in the steering portion 14 has various advantages including reducing the minimum bending radius achievable without kinking.

One advantage is that the lower friction surface of the spring 44 facilitates the pull wire 40 moving more freely through the steering portion 14, thus diminishing friction and drag effects in that area, and improving performance. Another advantage is that the spring 44 aids the steering portion 14 in returning to its original, straight position, after tension on the pull wire 40 is released. The spring 44 does not translate with the pull wire 40 when the pull wire 40 is pulled, and is stronger than the surrounding catheter material. The spring 44 provides a stronger surface area against which the pull wire 40 slides and pushes, and helps prevent the pull wire 40 from cutting through the concave side 33 of the steering portion 14 when the steering portion 14 is bent.

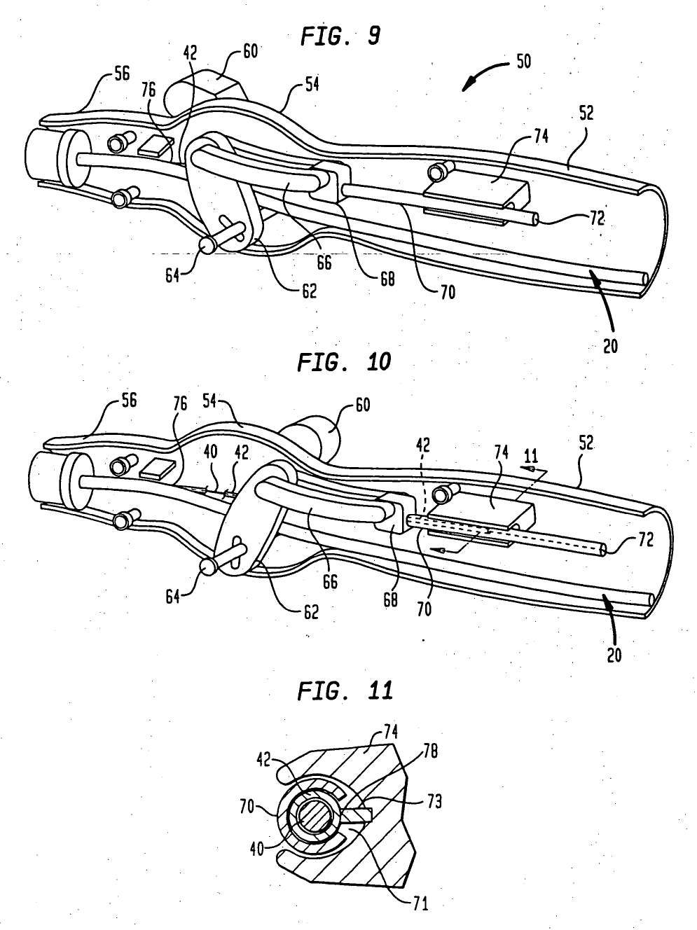

In accordance with yet another embodiment of the invention, the steering device 18 generally depicted in FIG. 1 may be in the form or a steering handle 50 provided at the proximal end 12 of the catheter 10, as depicted in FIGS. 9 and 10. The exterior shell, or housing, of the steering handle 50 comprises a proximal handle portion 52, intermediate handle portion 54 and distal handle portion 56. Preferably, the proximal handle portion 52 is shaped so as to conveniently fit in the palm of a user's hand. The intermediate portion 54 is shaped and oriented relative to the proximal portion 52 so that the user's fingers, and particularly the user's thumb, comfortably overlay it. The distal portion 56 houses and aligns the proximal catheter 20 with the intermediate 54 and proximal 52 portions of the handle 50.

Preferably, on the outside of the intermediate handle portion 54 is an outer movement element such as outer lever 60. Generally, in order to get the distal end 16 of the catheter 10 to bend, the outer lever 60 is moved proximally from a distal position, as depicted in FIG. 9, to a proximal position, as depicted in FIG. 10. This will be discussed in more detail, below.

The outer lever 60 is fixedly connected via connecting pin 64, which passes through intermediate handle portion 54, to an inner lever 62 such that movement of the outer lever 60 causes identical movement of the inner lever 62. Inner lever 62 is pivotally connected to piston rod 66, which is pivotally connected to piston 68. Piston 68, in turn is fixedly connected to an inner movement element such as guide rod 70. Guide rod 70 is in slidable frictional engagement with guide arm 74. Arm 74 is an internal extension of proximal handle portion 52 which constrains guide rod 70 and only permits longitudinal movement of the guide rod 70.

Guide tube 42 and the pull wire 40 contained within it, exit the catheter body at an exit point 76 on proximal catheter portion 20. Exit point 76 is disposed inside the handle 50, distal to the inner lever 62. Both the guide tube 42 and proximal catheter portion 20 pass through inner lever 62. The guide tube 42 remains unbent along its length from the exit point 76 all the way to its proximal end inside the proximal handle portion 52. The proximal catheter portion 20 bends slightly proximal to the exit point 76, and passes through the handle 50 to the exterior where it is available for common known catheter usage at that end.

The guide tube 42 is telescopically received within the piston 68 and guide rod 70. Preferably, as discussed above with reference to FIGS. 6-8, the guide tube 42 is also anchored in the pull wire lumen 38 at two anchor points 46 and 48, although more or less anchor points may be acceptable.

The guide tube 42 terminates within the guide rod 70 distal to the proximal end or terminus 72 of guide rod 70. The pull wire 40 extends proximally from the end of the guide tube 42 and continues within guide rod 70. The proximal end of the pull wire 40 is fixed to the guide rod 70 at the terminus 72 of the guide rod as, for example, by crimping or welding.

Steering of the catheter 10 occurs by moving the outer lever 60 from its distal position, as shown in FIG. 9, to a proximal position, as shown in FIG. 10. Such rotation of the outer lever 60 causes similar rotation of the inner lever 60, which pushes on the piston rod 66, which then translates the piston 68 and guide rod 70 proximally. Since the pull wire 40 is fixed to the guide rod's terminus 72, as the guide rod 70 is moved proximally, the pull wire 40 is pulled proximally. Because the guide tube 42 is anchored in the pull wire lumen 38 at two anchor points 46 and 48, it does not move when the guide rod 70 moves. Therefore, as the guide arm 74 moves proximally, the pull wire 40 gets pulled proximally relative to the guide tube 42 and relative to the catheter. This translational movement of the pull wire 40 exerts a pulling force on the distal portion of the catheter 10 where the pull wire 40 is anchored, thus causing the catheter to bend in the steering portion 14 as discussed above.

When the outer lever 60 is released from its proximal position as shown in FIG. 10, frictional forces between the outer lever and the intermediate handle portion 54 ensure stability of the deflected catheter position by holding the lever 60 in place. At the same time, the resilience and tendency for the catheter 10 to be in its unbent, unstressed condition, acts on the pull wire 40, so that when the outer lever 60 is moved to it's distal most setting as shown in FIG. 9, the distal end 16 of the catheter 10 returns to the straight position without requiring the pull wire 40 to provide any axial pushing forces. This is advantageous because it prevents the pull wire 40 from buckling in the handle 50 which can cause loss of full range deflection, or return of the distal end 16 of the catheter 10 to the straight position.

In this arrangement, the pull wire 40 is not made to pass over any sheaves or pulleys which stress and fatigue the pull wire 40 while changing its direction of travel. Rather, the pull wire 40 moves in a predominantly straight direction, and linearly and unobstructedly moves into and out of the proximal catheter portion 20 at point 76, thus minimizing any undesirable frictional forces acting upon it. This straight, unobstructed movement of the pull wire 40 enhances the responsiveness of the catheter 10 to steering forces applied at the steering handle 50. Further, the proximal end of the pull wire 40 moves in a straight path into and out of the proximal end of the guide tube 42. The telescopic relationship between the guide tube 42 and guide rod 70 assures such straight path.

In an alternative to the above configuration, the guide tube 42 may be fixed differently with respect to the guide rod 70. As best seen in FIG. 11, the guide rod 70 has an opening or longitudinal slot 71 that faces the guide arm 74. The guide tube 42 is anchored to the guide arm by an anchor element 73 which projects through slot 71 to the guide arm 74 at anchor point 78. In this arrangement, the guide tube 42 remains fixed while guide rod 70 freely moves proximally and distally over the guide tube 42.

The arrangement shown in FIG. 11 may be reversed. In such a reverse arrangement, the proximal end of the guide tube 42 is enlarged, and guide rod 70 moves within guide tube 42. The guide tube 42 remains attached to proximal handle portion 52. In this reversed arrangement, the connection between the guide rod 70 and the rest of the steering mechanism, such as the connection to piston 68, is located at a point proximal to the proximal end of the enlarged guide tube 42.

Additionally, other mechanical arrangements are envisioned as alternatives to the mechanism depicted in FIGS. 9 and 10. For example, the outer movement element or outer lever 60 may be in the form of a slider that moves in only one dimension, such as proximally and distally, rather than having a horizontal and vertical movement component as in the present case. Still further, a simplified linear pull mechanism may also be sufficient to telescopically move the guide rod 70 with respect to the guide tube 42.

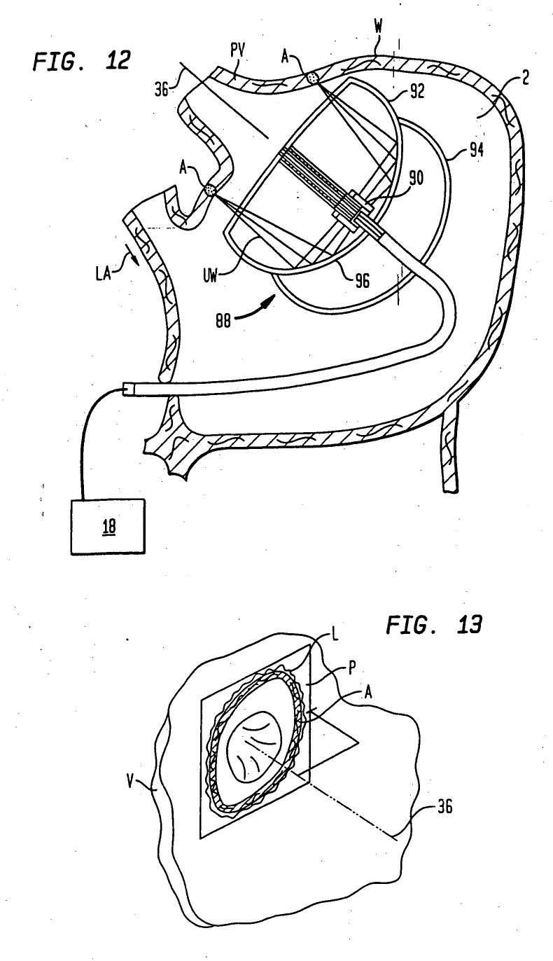

The instrument 17 disposed on the distal end of the catheter may be an ablation unit 88 as shown in FIGS. 12 and 13. The ablation unit 88 facilitates treating cardiac abnormalities, such as atrial fibrillation, by directing and focusing energy, such as ultrasound waves UW onto a region of the wall W of the heart to scar the cardiac tissue and disrupt electrical impulses between the pulmonary vein PV and the left atrium LA of the heart.

The ablation unit 88 comprises an ultrasonic emitter 90 attached to the catheter's distal end 16, and surrounded by a structural balloon 92. Proximal to the structural balloon is a reflector balloon 94. The structural balloon 92 and reflector balloon 94 are arranged such that they share a common wall 96.

Ultrasound waves UW emanating from the emitter 90 are deflected and focused by the common wall 96 into a ring in an ablation zone A which is generally located in a plane P that is perpendicular to the proximal-to-distal axis 36 of the catheter 10.

When the ablation unit 88 is steered into position in a heart chamber 2, such as the left atrium LA, and aligned to face the pulmonary vein PV such that the ablation zone A overlays the heart wall W, when the emitter 90 is actuated, a loop-like lesion L forms on the heart wall W in the ablation zone A. Such lesion, or scar, disrupts electrical impulses between the pulmonary vein PV and the left atrium LA of the heart, thus treating atrial fibrillation.

Further disclosure of such an ablation unit 88 and catheter, as well as the methods of its use, are provided in aforesaid U.S. Published Patent Application No. 2004/0176757, and U.S. Pat. No. 6,635,054, which have been fully incorporated by reference herein.

The various features discussed above optionally may be combined with one another. For example, a single device may include a catheter body having an oblique transition as discussed with reference to FIGS. 2-5; a guide tube as shown in FIGS. 6-8; a spring as also shown in FIGS. 6-8; and a steering mechanism as shown in FIGS. 9-11. Alternatively, the individual features can be used separately. For example, the spring can be used in the steering section of a catheter which does not incorporate the guide tube or oblique transition.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A steerable catheter comprising:
an elongated catheter body having a proximal-to-distal axis, said body including a proximal shaft portion and a distal shaft portion that is more flexible than said proximal shaft portion, said proximal and distal shaft portions joining on another at a junction;
said junction being generally oblique to said proximal-to-distal axis so that said proximal shaft portion terminates at an apex on a first side of said catheter and at a base on a second side diametrically opposite to said first side, said base being proximal to said apex.

2. The steering catheter of claim 1, wherein said apex of said proximal shaft portion overlays said distal shaft portion.

3. The steering catheter of claim 1, further comprising a steering device adapted to bend said distal shaft portion toward said first side of said catheter body, whereby said apex is oriented on a concave side of the bend when said steering portion is bent by said steering device.

4. The steering catheter of claim 3, wherein said steering device includes a pull wire extending generally proximally and distally within said steering catheter closer to said first side and farther from said second side.

5. The steering catheter of claim 4, further comprising a guide tube extending proximally to distally within at least a portion of said proximal shaft portion adjacent said first side, said pull wire extending within said guide tube.

6. The steering catheter of claim 5, further comprising a coil spring distal to said guide tube, said pull wire extending through said coil spring.

7. The steering catheter of claim 1, wherein said proximal shaft portion includes a main shaft portion and a transition portion distal to the main shaft portion, the transition portion being more flexible than the main shaft portion, whereby said junction is formed between said transition shaft portion and said distal shaft portion.

8. The steering catheter of claim 1, wherein said proximal shaft portion and said distal shaft portion have different material compositions.

9. The steering catheter of claim 1, wherein said proximal shaft portion and said distal shaft portion have different cross-sectional dimensions that are transverse to said proximal-to-distal axis.

10. The steering catheter of claim 1, further comprising an ablation unit attached to said distal shaft portion.

11. The steering catheter of claim 1, wherein said ablation unit is arranged to direct energy into a ring-like ablation area surrounding said proximal-to-distal axis that is distal to said distal shaft portion.

12. A steering catheter comprising:
an elongated catheter body having a pull wire lumen;
a guide tube;
a coil spring; and
a pull wire;
said guide tube and said spring being located inside said lumen;
said spring being distal to said guide tube; and
said pull wire running through said guide tube and said coil spring.

13. The steering catheter of claim 12, wherein said spring is located inside a steering portion of said steering catheter.

14. The steering catheter of claim 12, wherein a distal end of said guide tube contacts a proximal end of said spring.

15. The steering catheter of claim 12, wherein said guide tube is anchored to said steering catheter at two anchor points.

16. A steerable catheter unit, comprising:
a housing;
a catheter body having a main portion projecting in a distal direction from said housing;
a flexible guide tube extending in said main portion of said catheter body, said guide tube having a proximal section projecting out of said catheter body;
a pull wire slidably disposed within said guide tube, said pull wire having a distal end extending distally from said guide tube and connected to said catheter, said pull wire having a proximal end extending proximally from said guide tube;
an outer movement element movably mounted on an outside of said housing;
an inner movement element disposed in said housing and connected to said proximal end of said pull wire, said inner movement element being in telescopic relation to said proximal end of said guide tube, said inner movement element being linked to said outer movement element so that said inner movement element and said pull wire can be moved proximally by moving said outer movement element.

17. The steerable catheter unit of claim 16, wherein said pull wire travels in a substantially straight path within said housing.

18. The steerable catheter unit of claim 16, wherein movement of said inner movement element is guided by a guide arm in said housing.

19. The steerable catheter unit of claim 18, wherein said inner movement element is a rod.

20. The steerable catheter unit of claim 19, wherein said rod telescopically moves over said proximal end of said guide tube.

21. The steerable catheter unit of claim 20, wherein said guide tube is attached at at least one anchor point to said catheter body.

22. The steerable catheter unit of claim 16, wherein said guide tube is a hypotube.
